(19) United States
(12) Patent Application Publication
(10) Pub. No.: US 2011/0059986 A1
(43) Pub. Date: Mar. 10, 2011

(54) INTERMITTENT DOSING REGIMEN FOR OVERWEIGHT AND OBESE SUBJECTS

(76) Inventors: Marc Alois Celine Maria Engelen, Turnhout (BE); Dagmar Theo Coleta Maria Ghislain Hoeben, Mol (BE)

(21) Appl. No.: 12/947,960

(22) Filed: Nov. 17, 2010

Related U.S. Application Data
(63) Continuation of application No. 11/547,242, filed on Oct. 3, 2006.

(30) Foreign Application Priority Data
Apr. 9, 2004 (EP) 04101470.5

(51) Int. Cl. A61K 31/496; A61P 3/04

(52) U.S. Cl. 514/254.07

(57) ABSTRACT

The present invention concerns an intermittent dosing regimen for the treatment of obesity or the reduction of body weight wherein a pharmaceutical composition containing an apoB secretion/MTP inhibitor is administered to a subject in need thereof for a period of time, then withheld for a period of time, and again administered for a period of time. The intermittent regimen may be repeated depending on the response in the subject that is being sought.

The present invention concerns an intermittent dosing regimen for the treatment of obesity or the reduction of body weight wherein a pharmaceutical composition containing an apoB secretion/MTP inhibitor is administered to a subject in need thereof for a period of time, then withheld for a period of time, and again administered for a period of time. The intermittent regimen may be repeated depending on the response in the subject that is being sought.

The microsomal triglyceride transfer protein (MTP) catalyses the transfer of lipids such as triglycerides, cholesteryl esters and phosphatidylcholine between phospholipid surfaces. MTP is found in the liver and intestine, both organs which produce lipoproteins. MTP is necessary for the production of apolipoprotein B (apoB) containing plasma lipoproteins, in particular apoB-100 within the liver, and apoB-48 within the intestine. ApoB-100 is the main protein component of VLDL (very low density lipoproteins). ApoB-48 is the main protein component of chylomicrons. Compounds that inhibit MTP reduce the secretion of apoB-containing lipoproteins and therefore have the potential to decrease VLDL and triglyceride plasmatic levels, and also intestinal lipid absorption. High VLDL plasmatic levels are a major risk factor for atherosclerosis and coronary artery diseases. Hence an intermittent dosing regimen of the present invention using apoB secretion/MTP inhibitors may be useful in the prevention, management and treatment of obesity, diabetes mellitus, non-insulin dependent diabetes mellitus, coronary heart disease, pancreatitis, mixed dyslipidemia, hyperlipemia, post-prandial hyperlipemia, hypercholesterolemia, hypertriglyceridemia, osteoarthritis and atherosclerosis.

A variety of apoB secretion/MTP inhibitors are known to one of ordinary skill in the art. Although any apoB secretion/MTP inhibitor may be used in the intermittent dosing regimens of the present invention, generally preferred apoB secretion/MTP inhibitors include those compounds that are disclosed in, for example, European patent applications EP-0,643,057, EP-0,719,763, EP-0,753,517, EP-0,764,647, EP-0,765,878, EP-0,779,276, EP-0,779,279, EP-0,799,828, EP-0,799,829, EP-0,802,186, EP-0,802,188, EP-0,802,192, and EP-0,802,197; international patent applications WO-96/13499, WO-96/33193, WO-96/40640, WO-97/26240, WO-97/43255, WO-97/43257, WO-98/16526, WO-98/23593, WO-00/32582, WO-02/081460, WO-02/42271 and WO-02/20501; and U.S. Pat. No. 5,595,872; U.S. Pat. No. 5,646,162; U.S. Pat. No. 5,684,014; U.S. Pat. No. 5,712,279; U.S. Pat. No. 5,739,135 and U.S. Pat. No. 5,789,197.

A particular apoB secretion/MTP inhibitor is mitratapide which is the INN (International Non Proprietary Name) for the compound (−)-[2S-[2α, 4α(S*)]]-4-[4-[4-[4-[[2-(4-chlorophenyl)-2-[[(4-methyl-4H-1,2,4-triazol-3-yl)thio]methyl]-1,3-dioxolan-4-yl]methoxy]-phenyl]-1-piperazinyl]phenyl]-2,4-dihydro-2-(1-methylpropyl)-3H-1,2,4-triazol-3-one having the following structure.

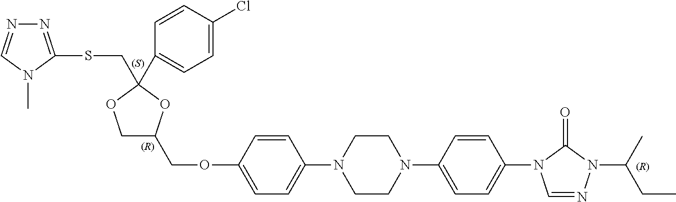

Mitratapide has been described in WO-96/13499 as compound (40) having apolipoprotein B (apoB) secretion and microsomal triglyceride transfer protein (MTP) inhibiting properties and therefore being useful as a lipid lowering agent.

Warm-blooded animals such as humans and companion animals, in particular dogs and cats, with an excessive accumulation of body fat to the point of being 20% or more over ideal body weight are considered obese. Already an overweight of 10% over ideal body weight is considered a health risk. Obesity is known to cause liver disease, hypertension, constipation, heat intolerance, and increased risk under anaesthesia. Obese warm-blooded animals may have trouble breathing and may suffer from serious discomfort and body dysfunction and have life expectancies less as usual. Although obesity in warm-blooded animals is usually caused by too little exercise and intake of too many calories, a number of warm-blooded animals become obese due to genetic predisposition or hormonal disorders.

Subjects suffering from obesity or overweight can be treated by administering an apoB secretion/MTP inhibitor. A pharmaceutical composition comprising the apoB secretion/MTP inhibitor is typically administered once or several times a day during a period of several weeks or months until the weight of the subject is equal to or close to its ideal body weight.

It has been observed that the administration of an apoB secretion/MTP inhibitor during a continuous period of eight weeks resulted in an initial reduction of body weight which however leveled off after three weeks. Sustained administration of the apoB secretion/MTP inhibitor did not result in a further reduction of body weight.

It has now been found that an intermittent treatment schedule or dosing regimen with alternating periods of administration and non-administration of the apoB secretion/MTP inhibitor can overcome the problem of body weight reduction levelling off. This intermittent treatment schedule or dosing regimen comprises of a period of several weeks during which the subject is administered an apoB secretion/MTP inhibitor followed by a period of several weeks of non-administration of the apoB secretion/MTP inhibitor, again followed by a period of several weeks of administration of the apoB secretion/MTP inhibitor. In order to achieve a further reduction of body weight, it is possible to repeat this intermittent treatment schedule two, three or four times.

For the purposes of this invention, the term “subject” includes warm-blooded animals, preferably mammals, including humans and companion animals such as dogs, cats, rabbits, ferrets, guinea pigs and the like.

The term “overweight” as used in the present invention refers to a body weight that is above the ideal body weight of a subject. Ideal body weight for human subjects can be determined using the “Body Mass Index” (BMI). The BMI is defined as the body weight in kilograms divided by the square of the height in meters. A BMI ranging from 20 to 25 is generally considered as ideal and human subjects having a BMI higher than 25 are considered overweight. Another method to determine ideal body for human subjects is based on the Metropolitan Life tables created by the Metropolitan Life Insurance company. Ideal body weight for companion animals, in particular dogs, can be looked up in breed standards, providing breed-specific information on body weight and height at withers for male and female animals.

The term “therapeutically effective amount of an apoB secretion/MTP inhibitor” as used herein, means that amount of an apoB secretion/MTP inhibitor that elicits the biological or medicinal response in the subject that is being sought, which includes alleviation of the symptoms of the condition being treated. The therapeutically effective amount can be determined using routine optimization techniques and is dependent upon the particular condition to be treated, the condition of the subject, the route of administration, the formulation, and the judgment of the practitioner and other factors evident to those skilled in the art. A therapeutically effective amount may be achieved by multiple dosing.

The regimen which is the basis of the present invention is an intermittent dosing regimen wherein a pharmaceutical composition containing an apoB secretion/MTP inhibitor is administered for a period of time, then withheld for a period of time, and again administered for a period of time. These three periods of time may be of the same or of different length. The length of each period can be expressed in days or in weeks and—dependent upon the specific apoB secretion/MTP inhibitor that is being used and the response of the subject—may range from 1 to 56 days or from 1 to 8 weeks. Said intermittent regimen may be repeated two, three, four or more times depending on the response in the subject that is being sought. The period of time between two intermittent dosing regimens is variable and in practice ranges from 2 to 6 months.

The intermittent dosing regimen consists of three terms which can be all of different length. Hence an infinite number of intermittent dosing regimens is possible by varying the length of each of the three terms. From a practical viewpoint it is preferable to express each term as a number of weeks so that one intermittent dosing regimen is defined as Aw-Bw-Cw wherein A represents the number of weeks during which an apoB secretion/MTP inhibitor is administered, B represents the number of weeks during which administration is withheld, and C represents the number of weeks during which an apoB secretion/MTP inhibitor is again administered. In practice, the first administration period ranges from 2 to 4 weeks, the period during which administration is withheld ranges from 2 to 4 weeks, and the second administration period ranges from 2 to 4 weeks. For instance, in a 4 w-3 w-4 w dosing regimen, the pharmaceutical composition comprising the apoB secretion/MTP inhibitor is administered for 4 weeks, withheld for 3 weeks, and again administered for 4 weeks. Practical dosing regimens are 4 w-4 w-4 w, 4 w-3 w-4 w, 4 w-2 w-4 w, 3 w-3 w-3 w, 3 w-2 w-3 w, and 2 w-2 w-2 w. The three terms of the intermittent dosing regimen may also be expressed in number of days.

The three terms of the intermittent dosing regimen may also be defined alternatively with a starting date and a final date. Accordingly a 4 w-3 w-4 w dosing regimen can be expressed as 1-28/29-49/50-77 which refers to administration of an apoB secretion/MTP inhibitor from day 1 to day 28, no administration from day 29 to day 49, and again administration from day 50 to day 77. The following table lists the above described practical dosing regimens expressed in weeks recalculated with a starting and final date.

| | Intermittent dosing regimen with starting and final dates | | | | | |
|---|---|---|---|---|---|---|
| | 1^{st} administration | | no administration | | 2^{nd} administration | |
| | period | | period | | period | |
| | Start date | Final date | Start date | Final date | Start date | Final date |
| 4 w-4 w-4 w | 1 | 28 | 29 | 56 | 57 | 84 |
| 4 w-3 w-4 w | 1 | 28 | 29 | 49 | 50 | 77 |
| 4 w-2 w-4 w | 1 | 28 | 29 | 42 | 43 | 70 |
| 3 w-3 w-3 w | 1 | 21 | 22 | 42 | 43 | 63 |
| 3 w-2 w-3 w | 1 | 21 | 22 | 35 | 36 | 56 |
| 2 w-2 w-2 w | 1 | 14 | 15 | 28 | 29 | 42 |

The present invention provides an intermittent dosing regimen for the treatment of obesity which is defined as Aweeks-Bweeks-Cweeks wherein A represents the number of weeks during which a pharmaceutical composition containing an apoB secretion/MTP inhibitor as the active ingredient in a therapeutically effective amount is administered to a subject in need thereof, B represents the number of weeks during which administration is withheld, and C represents the number of weeks during which said pharmaceutical composition containing the apoB secretion/MTP inhibitor is again administered. In practice, A ranges from 2 to 4 weeks, B ranges from 2 to 4 weeks and C ranges from 2 to 4 weeks. Practical dosing regimens are 4 w-4 w-4 w, 4 w-3 w-4 w, 4 w-2 w-4 w, 3 w-3 w-3 w, 3 w-2 w-3 w, and 2 w-2 w-2 w.

The present invention also provides an intermittent dosing regimen for the reduction of body weight which is defined as Aweeks-Bweeks-Cweeks wherein A represents the number of weeks during which a pharmaceutical composition containing an apoB secretion/MTP inhibitor as the active ingredient in a therapeutically effective amount is administered to a subject in need thereof, B represents the number of weeks during which administration is withheld, and C represents the number of weeks during which said pharmaceutical composition containing the apoB secretion/MTP inhibitor is again administered. In practice, A ranges from 2 to 4 weeks, B ranges from 2 to 4 weeks and C ranges from 2 to 4 weeks. Practical dosing regimens are 4 w-4 w-4 w, 4 w-3 w-4 w, 4 w-2 w-4 w, 3 w-3 w-3 w, 3 w-2 w-3 w, and 2 w-2 w-2 w.

Consequently there is provided the use of a pharmaceutical composition containing an apoB secretion/MTP inhibitor as the active ingredient in a therapeutically effective amount for the manufacture of a medicament for the treatment of obesity or the reduction of body weight wherein said pharmaceutical composition is administered according to an intermittent Aweeks-Bweeks-Cweeks regimen wherein A represents the number of weeks during which said pharmaceutical composition is administered to a subject in need thereof, B represents the number of weeks during which administration is withheld, and C represents the number of weeks during which said pharmaceutical composition is again administered. In practice, A ranges from 2 to 4 weeks, B ranges from 2 to 4 weeks and C ranges from 2 to 4 weeks. Practical regimens are 4 w-4 w-4 w, 4 w-3 w-4 w, 4 w-2 w-4 w, 3 w-3 w-3 w, 3 w-2 w-3 w, and 2 w-2 w-2 w.

Alternatively, an intermittent dosing regimen is provided for the treatment of obesity or the reduction of body weight comprising administering a pharmaceutical composition containing an apoB secretion/MTP inhibitor as the active ingredient in a therapeutically effective amount to a subject in need thereof on days 1 to 28, and on days 57 to 84. Other intermittent dosing regimens are administration on

a) days 1 to 28, and on days 50 to 77; or on
b) days 1 to 28, and on days 43 to 70; or on
c) days 1 to 21, and on days 43 to 63; or on
d) days 1 to 21, and on days 36 to 56; or on
e) days 1 to 14, and on days 29 to 42.

Consequently there is provided the use of a pharmaceutical composition containing an apoB secretion/MTP inhibitor as the active ingredient in a therapeutically effective amount for the manufacture of a medicament for the treatment of obesity or the reduction of body weight wherein said pharmaceutical composition is administered intermittently to a subject in need thereof on days 1 to 28, and on days 57 to 84. Other intermittent regimens are administration on

a) days 1 to 28, and on days 50 to 77; or on
b) days 1 to 28, and on days 43 to 70; or on
c) days 1 to 21, and on days 43 to 63; or on
d) days 1 to 21, and on days 36 to 56; or on
e) days 1 to 14, and on days 29 to 42.

According to a further aspect of the present invention there is also provided a pharmaceutical kit comprising dosage forms for administration to a subject in need thereof on days 1 to 28 and on days 57 to 84, which kit comprises dosage forms containing an apoB secretion/MTP inhibitor as the active ingredient in a therapeutically effective amount and a memory aid in the form of numbers or a calendar indicating on which days of the regimen the dosage forms should be ingested. The pharmaceutical kit may further comprise a patient information leaflet comprising the memory aid and further instructions concerning the intermittent dosing regimen. The memory aid may also be in the form of an electronic timing device with an LCD readout displaying the date that the last dosage forms has been taken and/or the date when the next dosage form is to be taken. Also provided is the same pharmaceutical kit suitable for administration to a subject in need thereof on

a) days 1 to 28, and on days 50 to 77; or on
b) days 1 to 28, and on days 43 to 70; or on
c) days 1 to 21, and on days 43 to 63; or on
d) days 1 to 21, and on days 36 to 56; or on
e) days 1 to 14, and on days 29 to 42.

During the administration periods of the intermittent dosing regimen the daily dosage of the apoB secretion/MTP inhibitor mitratapide may range between 0.1 mg per kg body weight and 5 mg per kg body weight, particular between 0.31 mg/kg and 1.25 mg/kg. In practice a daily dosage of 0.63 mg per kg body weight is used. It may be appropriate to administer the daily dose in the form of two or more sub-doses at appropriate intervals throughout the day.

The daily dosage of the apoB secretion/MTP inhibitor may be calculated daily during the administration periods on the basis of the body weight or it may be calculated once weekly at the start of each week during the administration periods. In practice, the daily dosage of the apoB secretion/MTP inhibitor is calculated once at the beginning of each administration period. Alternatively the daily dosage of the apoB secretion/MTP inhibitor may also be calculated once at the start on one intermittent dosing regimen and remain unchanged during the two administration periods.

The effect on body weight reduction of the intermittent dosing regimens of the present invention can be improved if the subject under treatment is altering its eating habits. For instance, a reduction of the caloric intake will likely have a beneficial effect on body weight reduction when a subject is undergoing treatment for obesity. The effect of the intermittent dosing regimen can be improved when a subject is following a maintenance diet whereby the caloric content of said diet equals the caloric

expenditure of the subject during any or all of the three periods of the intermittent dosing regimen. In practice, a subject may follow the first period of the intermittent dosing regimen without altering its eating habits and then switch to a maintenance diet at the beginning of the second period during which administration of the apoB secretion/MTP inhibitor is withheld, and continue with the same maintenance diet during the third period wherein the apoB secretion/MTP inhibitor is administered again. The caloric content of the maintenance diet is determined at the beginning of the second period and may be maintained or adapted during the remaining time of the intermittent dosing regimen. At the end of the second administration period a maintenance diet may be determined based on the weight of the subject in order to preserve the weight loss resulting from the intermittent dosing regimen.

In a further aspect of the present invention, a method for the reduction of body weight or the treatment of obesity of a subject in need thereof is provided wherein the intermittent dosing regimen is combined with a maintenance diet having a caloric content equal to the caloric expenditure of said subject. The maintenance diet may be followed concomitant with the beginning of the first, second or third period of the intermittent dosing regimen.

In another aspect, the intermittent dosing regimens of the present invention may be used in the cosmetic treatment of the human or animal body wherein the appearance of the human or animal body is improved by the loss of body weight. It may be desirable to obtain such a cosmetic improvement of bodily appearance by following an intermittent dosing regimen of the present invention.

The pharmaceutical compositions comprising an apoB secretion/MTP inhibitor can be administered to a subject either orally, parenterally (for example intravenously, intramuscularly or subcutaneously), percutaneously, or rectally.

Solid dosage forms for oral administration include capsules, dragees, tablets, powders and granules. These solid dosage forms are preferably formulated in dosage unit form for ease of administration and uniformity of dosage. “Dosage unit form” as used herein refers to physically discrete units suitable as unitary dosages, each unit containing a predetermined amount of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Liquid dosage forms for oral administration include pharmaceutically acceptable emulsions, solutions, suspensions, suspo-emulsions, syrups and elixirs. Pharmaceutical compositions for parenteral injection may comprise physiologically acceptable sterile aqueous or nonaqueous solutions, dispersions, suspension, or emulsions, or may comprise sterile powders for reconstitution into sterile injectable solutions or dispersions.

Pharmaceutical compositions comprising an apoB secretion/MTP inhibitor for administration to non-human animals can be administered in the drinking water so that a therapeutically effective amount is ingested with the daily water supply. The pharmaceutical compositions can also be added directly to the feed, as such, or in the form of an animal feed supplement, also referred to as a premix or concentrate.

The apoB secretion/MTP inhibitor may be used in conjunction with other pharmaceutical agents, in particular a lipid-lowering agent, thus leading to a so-called combination lipid-lowering therapy. The said additional lipid-lowering agent may be, for instance, a known drug conventionally used for the management of hyperlipidaemia such as e.g. a bile acid sequestrant resin, a fibric acid derivative or nicotinic acid. Suitable additional lipid-lowering agents also include other cholesterol biosynthesis inhibitors and cholesterol absorption inhibitors, especially HMG-CoA reductase inhibitors and HMG-CoA synthase inhibitors, HMG-CoA reductase gene expression inhibitors, CETP inhibitors, ACAT inhibitors, squalene synthetase inhibitors, CB-1 antagonists, cholesterol absorption inhibitors such as ezetimibe, and the like. The apoB secretion/MTP inhibitor and the other pharmaceutical agent for use in combination lipid-lowering therapy may be administered as separate dosage units or combined in one dosage unit.

Any HMG-CoA reductase inhibitor may be used as the second compound in the combination therapy aspect of this invention. The term “HMG-CoA reductase inhibitor” as used herein, unless otherwise stated, refers to a compound which inhibits the biotransformation of hydroxymethylglutaryl-coenzyme A to mevalonic acid as catalyzed by the enzyme HMG-CoA reductase. Such “HMG-CoA reductase inhibitors” are, for example, lovastatin, simvastatin, fluvastatin, pravastatin, rivastatin, and atorvastatin.

Any HMG-CoA synthase inhibitor may be used as the second compound in the combination therapy aspect of this invention. The term “HMG-CoA synthase inhibitor” as used herein, unless otherwise stated, refers to a compound which inhibits the biosynthesis of hydroxymethylglutaryl-coenzyme A from acetyl-coenzyme A and acetoacetyl-coenzyme A, catalyzed by the enzyme HMG-CoA synthase.

Any HMG-CoA reductase gene expression inhibitor may be used as the second compound in the combination therapy aspect of this invention. These agents may be HMG-CoA reductase transcription inhibitors that block the transcription of DNA or translation inhibitors that prevent translation of mRNA coding for HMG-CoA reductase into protein. Such inhibitors may either affect transcription or translation directly or may be biotransformed into compounds having the above-mentioned attributes by one or more enzymes in the cholesterol biosynthetic cascade or may lead to accumulation of a metabolite having the above-mentioned activities.

Any CETP inhibitor may be used as the second compound in the combination therapy aspect of this invention. The term “CETP inhibitor” as used herein, unless otherwise stated, refers to a compound which inhibits the cholesteryl ester transfer protein (CETP) mediated transport of various cholesteryl esters and triglycerides from HDL to LDL and VLDL.

Any ACAT inhibitor may be used as the second compound in the combination therapy aspect of this invention. The term “ACAT inhibitor” as used herein, unless otherwise stated, refers to a compound which inhibits the intracellular esterification of dietary cholesterol by the enzyme acyl CoA:cholesterol acyltransferase.

Any squalene synthetase inhibitor may be used as the second compound in the combination therapy aspect of this invention. The term “squalene synthetase inhibitor” as used herein, unless otherwise stated, refers to a compound which inhibits the condensation of two molecules of farnesylpyrophosphate to form squalene, catalyzed by the enzyme squalene synthetase.

The following examples describe the invention in greater detail and are intended to illustrate the invention.

### DESCRIPTION OF THE DRAWINGS

FIG. 1 is a graph displaying the results of an efficacy study wherein the apoB secretion/MTP inhibitor mitratapide was administered during 8 weeks to a group of obese Beagle dogs. The four curves illustrate the effect on body weight by plotting the ‘(%) body weight relative to the weight at the start’ when mitratapide was administered with a dosage of 0 mg per kg body weight (A curve), 0.16 mg per kg body weight (B curve), 0.31 mg per kg body weight (C curve) and 0.63 mg per kg body weight in function of the duration of the study.

FIG. 2 shows a graph displaying the results of a 4 w-4 w-4 w intermittent dosing regimen study using the apoB secretion/MTP inhibitor mitratapide. Mitratapide was administered for a first period of four weeks at a dosage of 0.63 mg/kg body weight, withheld for four weeks and again administered for four weeks at a dosage of 0.63 mg/kg body weight. At day 29, the feeding was restricted from ad libitum access to food, to a maintenance diet having a caloric content equal to the caloric expenditure of the test subject.

FIG. 3 shows a graph displaying the results of two intermittent dosing regimens: 3 w-2 w-3 w and 4 w-4 w-4 w including two placebo groups.

### EXPERIMENTAL PART

### Experiment 1

### Efficacy Study with Continuous Administration of Mitratapide During 8 Weeks

The efficacy of the apoB secretion/MTP inhibitor mitratapide for the reduction of body weight was studied in a blind, randomised study with 4 parallel groups of 6 dogs each. Three groups were treated orally with three different doses of mitratapide and one group was treated orally with the vehicle and served as a placebo group. The vehicle solution contained the same ingredients as the test formulations with omission of the test substance mitratapide.

The treatment groups were:

placebo group A treated orally with vehicle

group B treated orally with 0.16 mg mitratapide per kg body weight

group C treated orally with 0.31 mg mitratapide per kg body weight

group D treated orally with 0.63 mg mitratapide per kg body weight

The test subjects were healthy, male Beagle dogs with a body weight of 13.6 to 22.6 kg at the start of the experiment and between 1 and 8 years old. All dogs were treated orally using a 5 ml syringe, once daily, in the morning for a period of 8 weeks (56 days) depending on their body weight. Body weight was measured once weekly on days 0, 7, 14, 21, . . . up to day 56. The volume of test formulation was 1 ml per 4 kg body weight. The test formulation was an aqueous 10% hydroxypropyl-β-cyclodextrin solution containing no mitratapide, 0.63 mg mitratapide per ml, 1.25 mg mitratapide per ml or 2.5 mg mitratapide per ml. Each test subject had free access (in volume and time) to commercial dog feed (Bento Kronen Professional Power) and water during the 8 week study.

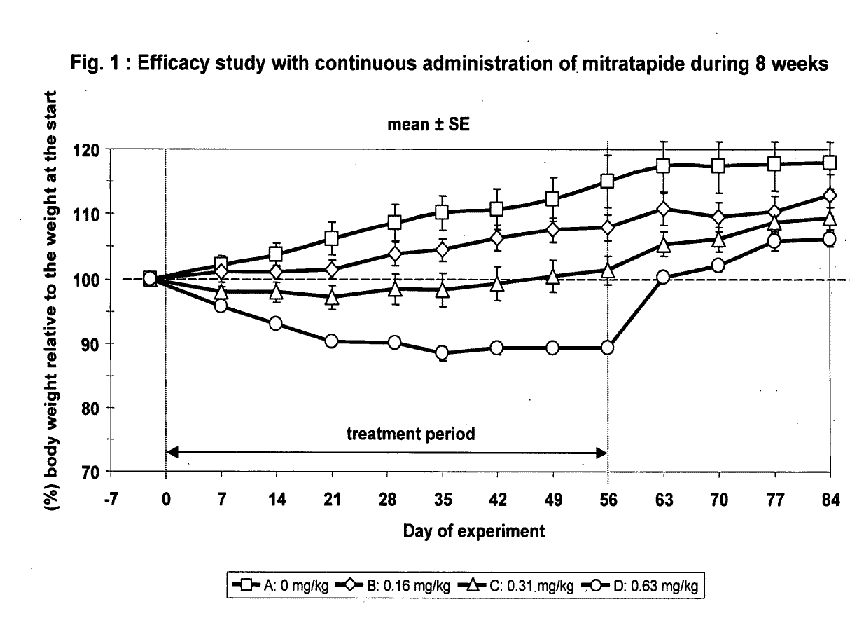

The effect on body weight for each of the four treatment groups is plotted in FIG. 1. As can be seen from FIG. 1, a daily dosage of at least 0.31 mg/kg was necessary to decrease body weight. A daily dosage of 0.63 mg/kg was more effective in reducing body weight. As illustrated in curve D, the reduction of body weight started to level off after three weeks and a further administration of mitratapide for the remaining five weeks did not result in a further reduction of body weight. This study clearly demonstrates the problem of a body weight reduction levelling off when an apoB secretion/MTP inhibitor is administered for a continuous period.

### Experiment 2

### 4 w-4 w-4 w Intermittent Dosing Regimen Study

The effect of a 4 w-4 w-4 w intermittent dosing regimen on 15 obese Beagle dogs, with a body weight of 16% or more above optimal body weight, was studied. The dogs were treated with mitratapide oral solution at a daily single dose of 0.63 mg/kg body weight for two periods of 28 consecutive days (4 weeks) with an intermediate period of 28 consecutive days without treatment.

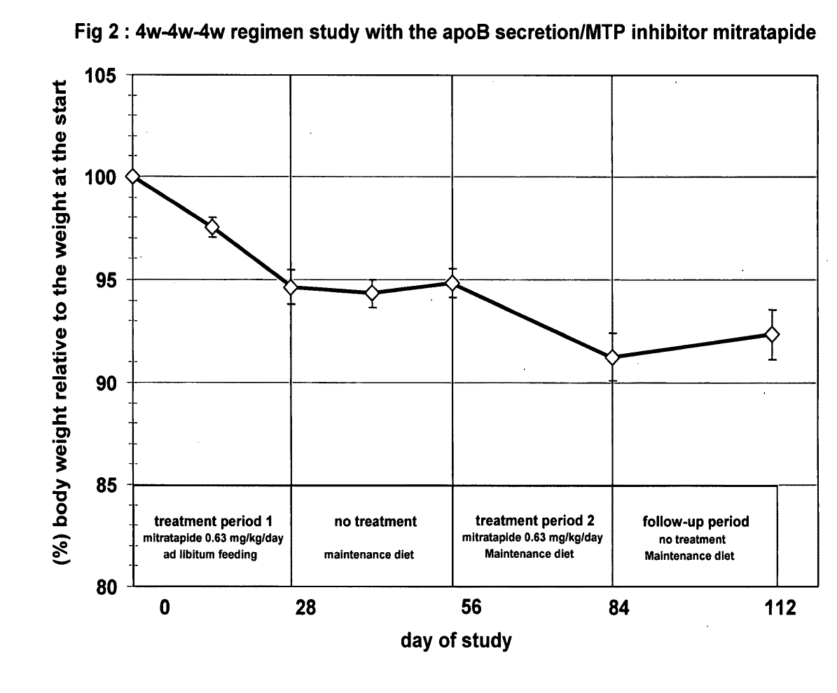

The test formulation comprising mitratapide was an aqueous 10% hydroxypropyl-β-cyclodextrin solution containing 2.5 mg mitratapide per ml and was administered once daily in an amount of 1 ml per 4 kg body weight. The daily dose was mixed into a small portion of feed and presented to the dog. The rest of the feed was only provided after this portion was consumed.
Each test subject had free access (in volume and time) to commercial dog feed during the first period of four weeks.
This first period of four weeks was followed by a period of four weeks during which administration of mitratapide was withheld. At the same time, the dogs were put on a maintenance diet having a caloric content equal to their caloric expenditure. The caloric content of the maintenance diet was calculated by multiplying the resting energy requirement (RER) for a dog, which is 290 kJ (=70 kcal) per kg metabolic weight, with a factor of 1.4 to obtain the daily energy requirement of an inactive dog. At day 56 of the study, the dogs were again administered once daily a mitratapide solution with a dosage of 0.63 mg/kg body weight for a period of four weeks. The dogs were kept on the same maintenance diet as during the previous period.
After 84 days the dogs were put on a new maintenance diet which was adjusted in accordance with their new body weight. The test solution comprising mitratapide was no longer administered and the body weight of the animals was further monitored during the follow-up procedure ending on day 112.
All dogs had free access to drinking water throughout the entire study.
Body weight of each test animal was measured on days 0, 14, 28, 42, 56, 84 and 112. The effect on body weight for each of the four dosage studies is plotted in FIG. 2. As can be seen from FIG. 2, a reduction of body weight was observed during the two treatment periods and no body weight reduction levelling off effect was observed. After 84 days, the mean reduction of body weight was 9%.

### Experiment 3

### Comparison of 3 w-2 w-3 w and 4 w-4 w-4 w Dosing Regimen

Thirty two healthy obese Beagle dogs of both sexes were included in a study of two different intermittent treatment regimens: a 3 w-2 w-3 w regimen and a 4 w-4 w-4 w regimen. At the start of the study, the dogs weighed between 12.5 and 26 kg which was considered as being more than 20% higher than the ideal body weight. The study was conducted in four groups, two treated groups containing 10 obese Beagle dogs and one placebo group containing 12 obese Beagle dogs. This placebo group of 12 dogs was subdivided in two sets of 6 dogs. One placebo group followed the 3 w-2 w-3 w regimen and the other placebo group the 4 w-4 w-4 w regimen.

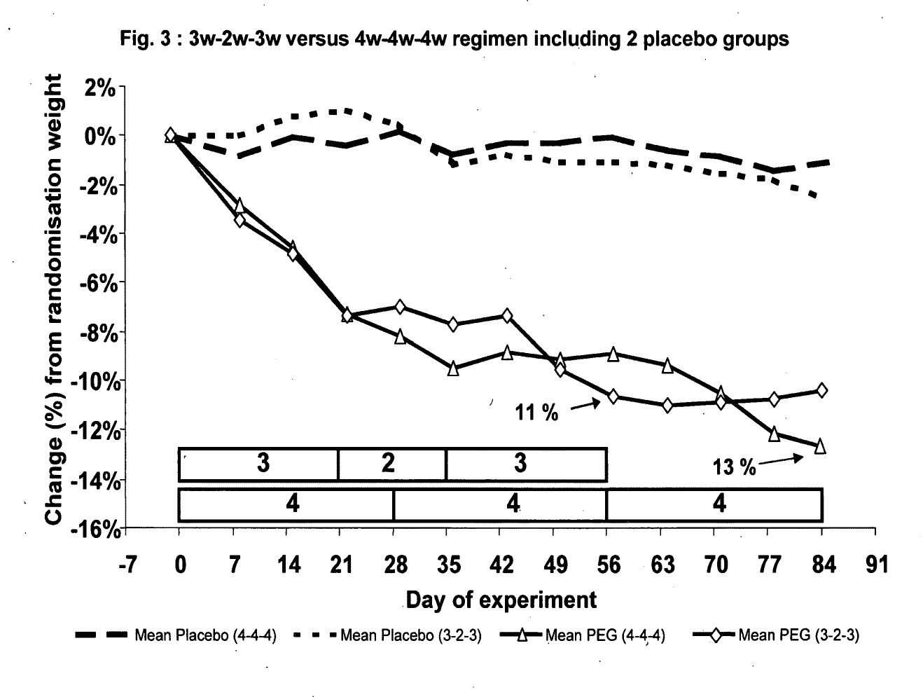

The test compound mitratapide was provided as a polyethylene glycol 400 (PEG 400) solution comprising 5 mg mitratapide per ml. The test solution was administered once daily in the morning by oral gavage. The volume of test formulations was 0.125 ml per kg body weight so that mitratapide was administered with a dosage of 0.63 mg/kg body weight. The body weight was measured weekly during the treatment periods to adjust the amount of test formulation. The placebo groups received water as the test formulation.
Each test subject had free access (in volume and time) to commercial dog feed (Bento Kronen premium Regular dog pellets) during the first administration period.
The first administration period was followed by a period during which administration of mitratapide was withheld. At the same time, the dogs were put on a maintenance diet having a caloric content equal to their caloric expenditure. The caloric content of the maintenance diet was calculated by multiplying the resting energy requirement (RER) for a dog, which is 290 kJ (=70 kcal) per kg weight, with a factor of 1.8 to obtain the daily energy requirement of an inactive dog.
At the start of the second administration period, the dogs were again administered a mitratapide solution with a dosage of 0.63 mg/kg body weight. The dogs were kept on the same maintenance diet as during the previous period.
All dogs had free access to drinking water throughout the entire study.
Body weight of each test animal was measured weekly on days 0, 7, 14, 21 and so on till day 84. The effect on body weight for each of the four dosage studies is plotted in FIG. 3. As can be seen from FIG. 3, a reduction of body weight was observed for the two intermittent dosing regimens and no body weight reduction levelling off effect was observed. The reduction of body weight of the two placebo groups was less than 2% after 84 days.
The 3 w-2 w-3 w dosing regimen had a mean effect on body weight reduction of 11% at the end of the dosing regimen study on day 56.
The 4 w-4 w-4 w dosing regimen had a mean effect on body weight reduction of 13% at the end of the dosing regimen study on day 84.

1. An intermittent dosing regimen for the treatment of obesity or the reduction of body weight wherein a pharmaceutical composition containing an apoB secretion/MTP inhibitor as the active ingredient in a therapeutically effective amount is administered to a subject in need thereof for Aweeks-Bweeks-Cweeks wherein A ranges from 2 to 4 and represents the number of weeks during which a pharmaceutical composition containing an apoB secretion/MTP inhibitor as the active ingredient in a therapeutically effective amount is administered to a subject in need thereof, B ranges from 2 to 4 and represents the number of weeks during which administration is withheld, and C ranges from 2 to 4 and represents the number of weeks during which said pharmaceutical composition containing the apoB secretion/MTP inhibitor is again administered.

2-3. (canceled)

4. A regimen as claimed in claim 1 wherein Aweeks-Bweeks-Cweeks is selected from 4 w-4 w-4 w, 4 w-3 w-4 w, 4 w-2 w-4 w, 3 w-3 w-3 w, 3 w-2 w-3 w, and 2 w-2 w-2 w.

5. A regimen as claimed in claim 4 wherein Aweeks-Bweeks-Cweeks is 3 w-2 w-3 w.

6. A regimen according to claim 1 wherein the apoB secretion/MTP inhibitor is mitratapide.

7. A regimen according to claim 6 wherein mitratapide is administered in a daily amount ranging from 0.31 mg per kg body weight to 1.25 mg per kg body weight.

8. A regimen according to claim 7 which is combined with a maintenance diet having a caloric content equal to the caloric expenditure of the subject.

9. A regimen according to claim 8 wherein the subject is a companion animal.

10. A pharmaceutical kit comprising dosage forms for administration to a subject in need thereof on days 1 to 28 and on days 57 to 84; or on days 1 to 28, and on days 50 to 77; or on days 1 to 28, and on days 43 to 70; or on days 1 to 21, and on days 43 to 63; or on days 1 to 21, and on days 36 to 56; or on days 1 to 14, and on days 29 to 42, which kit comprises dosage forms containing an apoB secretion/MTP inhibitor as the active ingredient in a therapeutically effective amount and a memory aid in the form of numbers or a calendar indicating on which days of the regimen the dosage forms should be ingested.

11. A method of treating obesity, comprising administering a pharmaceutical composition containing an apoB secretion/MTP inhibitor as the active ingredient in a therapeutically effective amount according to an intermittent Aweeks-Bweeks-Cweeks regimen wherein A ranges from 2 to 4 weeks and represents the number of weeks during which said pharmaceutical composition is administered to a subject in need thereof, B ranges from 2 to 4 weeks and represents the number of weeks during which administration is withheld, and C ranges from 2 to 4 weeks and represents the number of weeks during which said pharmaceutical composition is again administered.

12. The method according to claim 11 wherein Aweeks-Bweeks-Cweeks is selected from 4 w-4 w-4 w, 4 w-3 w-4 w, 4 w-2 w-4 w, 3 w-3 w-3 w, 3 w-2 w-3 w, and 2 w-2 w-2 w.

13. The method according to claim 12 wherein Aweeks-Bweeks-Cweeks is 3 w-2 w-3 w.

14. The method according to claim 11 wherein the apoB secretion/MTP inhibitor is mitratapide.

15. The method according to claim 14 wherein mitratapide is administered in a daily amount ranging from 0.31 mg per kg body weight to 1.25 mg per kg body weight.

16. A regimen as claimed in claim 1, wherein the regimen is for the reduction of body weight.

17. A regimen as claimed in claim 7, wherein mitratapide is administered in a daily amount of 0.63 mg per kg body weight.

18. A regimen according to claim 9, wherein the companion animal is a dog or a cat.
